SEQUENCE LISTING (1 sequence)

SEQ ID NO: 1 - DNA, 4484 bp, Artificial Sequence
  PGK1p-PYC2-PGK1t synthetic construct
aagcttggccggccgggcccttaccaataatgtttaatgtatatatatatatatatatatggggccgtatacttacatatagtagatgtcaagcgtaggcgcttcccctgccggctgtgagggcgccataaccaaggtatctatagaccgccaatcagcaaactacctccgtacattcatgttgcacccacacatttatacacccagaccgcgacaaattacccataaggttgtttgtgacggcgtcgtacaagagaacgtgggaactttttaggctcaccaaaaaagaaagaaaaaatacgagttgctgacagaagcctcaagaaaaaaaaaattcttcttcgactatgctggaggcagagatgatcgagccggtagttaactatatatagctaaattggttccatcaccttcttttctggtgtcgctccttctagtgctatttctggcttttcctatttttttttttccatttttctttctctctttctaatatataaattctcttgcattttctatttttctctctatctattctacttgtttattcccttcaaggtttttttttaaggagtacttgtttttagaatatacggtcaacgaactataattaactaaacatgtcctcttccaagaaattggccggtttgagagacaacttctctttgttgggtgaaaagaacaaaatcttggttgctaacagaggtgaaatcccaatcagaattttcagatctgctcatgaactatccatgagaaccattgctatctactctcatgaagatcgtttgtccatgcacagattgaaggctgatgaggcttacgttattggtgaagaaggtcaatacactccagttggtgcctacttggccatggacgaaattattgaaattgccaagaagcacaaggtcgatttcatccacccaggttacggtttcttgtccgaaaactctgaatttgctgacaaggttgtcaaggctggtatcacttggatcggtccaccagctgaagtcattgactctgtcggtgacaaagtctctgctcgtcatttggctgctcgtgccaatgtcccaactgttccaggtactccaggtccaattgaaactgttcaagaagccttggatttcgtcaatgaatacggttacccagttatcatcaaggccgctttcggtggtggtggtcgtggtatgagagttgttagagaaggtgatgacgttgctgatgctttccaaagagctacctctgaagccagaactgctttcggtaacggtacctgtttcgtcgaaagattcttggacaagccaaagcacattgaagttcaattgttggctgacaaccacggtaacgttgtccatttgttcgaaagagactgttctgttcaacgtcgtcaccaaaaggttgttgaagttgctccagccaagactttaccaagagaagtccgtgatgctatcttgactgacgctgtcaaattagctaaggtttgtggttacagaaacgctggtactgctgaattcttggttgacaaccaaaacagacattatttcatcgaaattaacccaagaattcaagtcgaacacactatcactgaagaaatcactggtattgacattgtctctgctcaaatccaaattgctgctggtgccactttgactcaattgggtttgttgcaagacaagatcaccactagaggtttctccatccaatgtcgtatcactaccgaggacccatccaagaacttccaaccagacaccggtcgtttggaagtttacagatctgctggtggtaacggtgtcagattggatggtggtaacgcttacgctggtgctactatctctcctcactacgactccatgttggtcaaatgttcctgttccggttccacttacgaaattgtcagaagaaagatgatcagagctttgatcgaattcagaatcagaggtgttaagaccaacattccatttttgttgactttgttgaccaaccctgttttcattgaaggtacttactggaccactttcattgacgacactccacaattgttccaaatggtttcctctcaaaacagagctcaaaaattgttgcactacttggctgatttggctgtcaacggttcttctatcaagggtcaaattggtttaccaaagttgaaatctaacccttccgttccacacttgcacgacgctcaaggtaacgtcatcaacgttactaaatctgctccaccatctggttggagacaagttttgttggaaaagggtccatctgaatttgccaagcaagtcagacaattcaacggtactttgttgatggacaccacctggagagatgctcaccaatctttattggctaccagagttagaacccacgatttggctactatcgctccaaccaccgctcatgccttggccggtgctttcgccttggaatgttggggtggtgccaccttcgatgtcgccatgagattcttgcacgaggacccatgggaaagactaagaaagttgagatctttggttccaaacattccattccaaatgttgctaagaggtgctaacggtgttgcttactcttctttaccagacaatgccattgaccacttcgtcaagcaagctaaggacaacggtgttgacatcttcagagttttcgatgctttgaatgacttggaacaattaaaggtcggtgttaacgctgtcaagaaggctggtggtgttgttgaagctaccgtttgttactccggtgacatgttgcaacctggtaagaagtacaacttggactactacttggaagttgttgaaaagatcgtccaaatgggtacccacatcttaggtatcaaggacatggctggtaccatgaagccagctgctgctaagttgttgatcggttctttgagaaccagatacccagatttgccaattcacgtccactctcacgattctgccggtactgccgttgcttccatgactgcttgtgctttggctggtgctgatgtcgtcgatgttgccatcaactccatgtctggtttgacctctcaaccatccattaacgctttgttggcctccttggaaggtaacatcgacaccggtatcaacgtcgaacacgttagagaattagatgcttactgggctgaaatgagattattgtactcttgtttcgaagctgacttgaagggtccagacccagaagtctaccaacacgaaattccaggtggtcaattgaccaacttattgttccaagctcaacaattgggtttaggtgaacaatgggctgaaaccaagagagcttacagagaagccaactacttgttgggtgacattgtcaaggttactccaacctccaaggttgttggtgacttggcccaattcatggtcagcaacaaattaacctctgatgacatcagaagattggctaactctctagacttcccagattccgtcatggacttctttgaaggtttgatcggtcaaccatacggtggtttcccagaaccattacgttctgacgtcttaagaaacaagagaagaaagttgacttgtagaccaggtttggaattggaaccatttgacttggaaaagatcagagaagatttgcaaaacagattcggtgatattgacgaatgtgatgttgcttcttacaacatgtacccaagagtttacgaagatttccaaaagatcagagaaacctacggtgatttgtccgtcttaccaaccaagaactttttggccccagctgaaccagatgaagaaattgaagtcaccattgaacaaggtaagactttgatcatcaaattacaagctgttggtgacttgaacaagaaaaccggtcaaagagaagtctacttcgaattaaacggtgaattgagaaagatcagagttgctgacaaatctcaaaacatccaatccgttgccaagccaaaggctgatgttcacgacacccaccaaatcggtgctccaatggctggtgttatcatcgaagtcaaggtccacaagggttccttggtcaagaagggtgaatctattgctgttttgtccgctatgaagatggaaatggttgtctcttctccagctgacggtcaagtcaaggacgttttcatcaaagatggtgaatccgtcgatgcttctgatttgttggttgtcttggaagaagaaactttgcctccatcccaaaagaagtaaggagtaataattattgcttccatataatatttttatatacctcttatttttatgtattagttaattaagtatttttatctatctgcttatcattttcttttcatatagggggggttggtgttttcttgcccatcagattgatgtcctccaactcggcactattttacaaagggtttttttgtaagagaaggagaagacagatactaaaccatacgttactcgaaacaaaaaaaaaaaaaatggaaaaagctgctatcaacaaaagacggcctcatcaaacctaaagaaaccatgtcagcgtggatccgcggccgcacgcgt

(19) United States
(12) Patent Application Publication
(10) Pub. No.: US 2012/0238722 A1
(43) Pub. Date: Sep. 20, 2012

(54) PROCESS FOR THE CRYSTALLIZATION OF SUCCINIC ACID

(75) Inventors: Maarten Job Van De Graaf, Zoetermeer (NL); Fredoen Vallianpoer, Amsterdam (NL); Guillaume Fiey, Lille (FR); Loic Delattre, Hinges (FR); Elisabeth Agnes Michèle Schulten, Den Haag (NL)

(73) Assignees: ROQUETTE FRERES SA, Lestrem (FR); DSM IP ASSETS B.V., Heeleen (NL)

(21) Appl. No.: 13/511,365

(22) PCT Filed: Nov. 19, 2010
(86) PCT No.: PCT/EP2010/067868

§ 371 Date: Jun. 1, 2012

Related U.S. Application Data
(60) Provisional application No. 61/263,968, filed on Nov. 24, 2009.

(30) Foreign Application Priority Data
Nov. 24, 2009 (EP) 09176923.2

(51) Int. Cl. C07C 51/43; C07C 55/10; C12P 7/46; C08G 63/16

(52) U.S. Cl. 528/272; 562/593; 562/590; 435/145

(57) ABSTRACT

The present invention relates to a process for recovering succinic acid in crystal form from a fermentation broth comprising succinic acid, comprising the steps of
a) bringing the fermentation broth to a pH of between 1 and 4,
b) crystallizing the succinic acid from the fermentation broth to form succinic acid crystals,
c) dissolving the succinic acid crystals at a temperature of between 30 and 90 degrees Celsius to form an aqueous solution comprising dissolved succinic acid,
d) crystallizing the succinic acid from the solution to recover succinic acid in crystal form.

The invention further relates to succinic acid in crystal form, comprising a sugar content of 1 to 100 ppm and a nitrogen content of 1 to 80 ppm.

The present invention relates to a process for the crystallization of succinic acid and to high purity succinic acid in crystal form.

Succinic acid is a C4-dicarboxylic acid and is an intermediate of the tricarboxylic acid cycle (TCA). Succinic acid finds numerous applications in the cosmetic, pharmaceutical, and food industry and as a monomer in the preparation of polyester polymers. Thus far, succinic acid is predominantly produced via petrochemical processes which are considered harmful to the environment. The biological production of succinic acid via fermentation has been focussed on as an attractive alternative to petrochemical-based processes. Various bacteria are known to produce succinic acid by fermentation such as Actinobacillus succinogenes, Mannheimia succiniciproducens and Escherichia coli, as well as fungi such as Aspergillus niger and Saccharomyces cerevisiae. Following fermentative production, succinic acid is recovered from a fermentation broth. Various processes for the recovery of succinic acid have been developed.

U.S. Pat. No. 5,168,055 discloses a process for the recovery of succinic acid from a fermentation broth containing calcium succinate, wherein sulphuric acid was used to convert succinate into succinic acid, and wherein calcium sulphate is produced. The succinic acid was further purified with a strong acidic cation exchange resin and a weak basic anion exchange resin. However, these purification steps, do not remove all coloring in the succinic acid crystals.

U.S. Pat. No. 6,265,190 discloses a process for the recovery of succinic acid from a fermentation medium comprising succinate, by the addition of ammonium sulphate. The succinic acid crystals were further purified by dissolution in methanol. Use of methanol for purifying succinic acid crystals is undesirable because of its toxicity and remaining traces of methanol in the succinic acid crystals in various applications are undesirable.

WO2009/082050 discloses a method for purifying succinic acid from a microbial culture broth, wherein prior to concentrating and acidifying the culture broth with hydrochloride and sulphuric acid, the culture broth is decolorized with activated carbon. A disadvantage of decoloring the whole culture broth is that a large amount of activated carbon is needed, which is expensive and not economical.

Another disadvantage of the processes disclosed in U.S. Pat. No. 5,168,055, U.S. Pat. No. 6,265,190 and WO 2009/082050 is that salts are added to acidify the fermentation broth and convert succinate to succinic acid, which are costly and impose additional process steps to recover succinic acid.

As an alternative to the addition of salts to convert succinate into succinic acid, U.S. Pat. No. 5,034,105 and U.S. Pat. No. 5,143,834 disclose a process for crystallizing succinic acid from a microbial culture broth by acidifying the culture broth using water-splitting or water-dissociation technology, after treatment of the culture broth with conventional electrodialysis. A disadvantage of this process is that two successive electrodialyses steps are applied which are costly and reduce succinic acid yield.

The aim of the present invention is an improved process for the recovery of succinic acid from a fermentation broth, wherein succinic acid is recovered at a high yield and purity, which overcomes the disadvantages outlined above.

The aim is achieved according to the present invention with a process for recovering succinic acid in crystal form from a fermentation broth comprising succinic acid, comprising bringing the fermentation broth to a pH of between 1 and 4, crystallizing the succinic acid to form succinic acid crystals from the fermentation broth, dissolving the succinic acid crystals at a temperature of between 30 and 90 degrees Celsius to obtain an aqueous solution of dissolved succinic acid, and crystallizing the succinic acid from the aqueous solution to obtain succinic acid in crystal form.

A first advantage of the process according to the present invention is that by dissolving the succinic acid crystals at a temperature of between 30 and 90 degrees Celsius, a lower amount of water needs to be evaporated during a subsequent crystallization step, resulting in a lower energy consumption, as compared to a process wherein succinic acid crystals are dissolved at a temperature below 30 degrees Celsius.

A second advantage of the process according to the present invention is that a smaller volume of a succinic acid solution can be treated in a subsequent decoloring treatment, as compared to processes for purifying succinic acid in crystal form known in the art, for instance the process disclosed in WO2009/082050. This results in lower costs for decoloring treatment.

A third advantage of the process according to the invention is, that it was found that by dissolving the succinic acid crystals at a temperature of between 30 and 90 degrees Celsius a higher amount of color was removed in a subsequent decoloring step, as compared to a process wherein the crystals were dissolved below a temperature of 30 degrees Celsius.

In a preferred embodiment, the process according to the present invention further comprises a step of decoloring the solution comprising dissolved succinic acid crystals. Decoloring may be performed in any suitable way such as by cation exchange chromatography or activated carbon. Decoloring may comprise treating the solution comprising dissolved succinic acid crystals with activated carbon. We found that there was a correlation between the amount of nitrogen and sugar present in the succinic acid in crystal form and the coloration. It was surprisingly found that after dissolution of succinic acid crystals at a temperature of between 30 and 90 degrees Celsius, treating the solution comprising dissolved succinic acid with activated carbon removed a higher amount of nitrogen and sugar from the succinic acid crystals, as compared to a process wherein the succinic acid crystals were dissolved below 30 degrees Celsius. Usually, decoloring the solution comprising succinic acid is followed by a step of subjecting the solution to ion exchange resin. Ion exchange resin may be cation- and/or anion-exchange resin known to the skilled man in the art, and which may be carried out in any suitable order.

Surprisingly, it was found that the process according to the present invention comprising a step of decoloring the solution comprising dissolved succinic acid crystals resulted in succinic acid in crystal form with a high purity. High purity as used herein is defined as succinic acid crystals comprising a sugar content of 1 to 100 ppm and a nitrogen content of 1 to 80 ppm. It was surprisingly found that succinic acid in crystal form obtained by the process according to the present invention showed little to no coloration. Little to no coloration as used herein is defined as succinic acid in crystal form having an absorption at 425 nm of between 0.001 and 0.150, as measured in a solution of succinic acid in butanediol at a ratio of succinic acid in crystal form:butanediol of 1:4 which has been heated at 180 degrees Celsius for 3 hours.

Crystallizing succinic acid from the fermentation broth in the process according to the present invention may be performed by any suitable method known in the art, such as crystallizing by bringing the temperature of the fermentation broth to a temperature of between 1 and 25 degrees Celsius, or evaporative crystallization. Crystallizing succinic acid in the process according to the present invention may comprise bringing the fermentation broth to a temperature of between 1 and 25 degrees Celsius, for instance between 2 and 20 degrees Celsius. A step of bringing the fermentation broth to a temperature of between 50 and 100 degrees Celsius, for instance between 60 and 80 degrees Celsius, or between 65 and 75 degrees Celsius precedes a step of bringing the fermentation broth to a temperature of between 1 and 25 degrees Celsius, The step of bringing the fermentation broth to a temperature of between 50 and 100 degrees Celsius is advantageous since this resulted in a fermentation broth with a higher concentration of succinic acid.

In one embodiment crystallizing succinic acid to form succinic acid crystals in the process according to the present invention may comprise separating succinic acid crystals from the fermentation broth, resulting in a mother liquor and succinic acid crystals. The separating of succinic acid crystals may be carried out by any suitable method known in the art, for instance by centrifugation or filtration. Crystallizing succinic acid to form succinic acid crystals may comprise a step of washing succinic acid crystals. The mother liquor may be recycled to a step of bringing the fermentation broth to a temperature of between 50 and 100 degrees Celsius as described herein above. A step of recycling the mother liquor was found advantageous to increase the yield of succinic acid.

Dissolving succinic acid crystals in the process according to the present invention may be carried out at a temperature of between 30 and 90 degrees Celsius, for instance between 35 and 85 degrees Celsius, or between 40 and 80 degrees Celsius. It was found advantageous that the dissolving of succinic acid crystals is carried out at a temperature of between 30 and 90 degrees Celsius, since a higher amount of succinic acid is dissolved in a lower amount of water as compared to dissolving succinic acid at a temperature of below 30 degrees Celsius. It was found advantageous to limit water consumption in the process according to the present invention, since this reduces the amount of energy and steam needed to evaporate water during subsequent crystallization of succinic acid from the solution comprising dissolved succinic acid. Succinic acid may be dissolved at a concentration of at least 200, 250, 300, 350, or 400 g/l or more, depending on the temperature at which succinic acid is dissolved. The maximum solubility of succinic acid at certain temperatures is known to the skilled person in the art.

Crystallizing succinic acid from a solution comprising dissolved succinic acid to obtain succinic acid in crystal form in the process according to the present invention may be carried out according to any crystallization method known in the art. For instance, crystallizing succinic acid from an aqueous solution comprising dissolved succinic acid may be carried out by bringing the temperature of the fermentation broth to a temperature of between 1 and 25 degrees Celsius as outlined herein above.

Crystallizing succinic acid from a solution comprising dissolved succinic acid to obtain succinic acid in crystal form, may comprise separating succinic acid in crystal form from the aqueous solution resulting in succinic acid crystals and a mother liquor. Said crystallizing may comprise washing and drying succinic acid in crystal form. Washing succinic acid in crystal form may comprise washing with water. The mother liquor from the aqueous solution may further be recycled to the step of dissolving succinic acid crystals at a temperature of between 30 and 90 degrees Celsius. It was found that recycling the mother liquor increased the overall yield of succinic acid, preferably to about 80, 85, or 90% of the succinic acid originally present in the fermentation broth.

In one embodiment, the fermentation broth in the process according to the present invention comprises salt components at a concentration of between 1 to 80 wt % of succinic acid, preferably between 5 and 70 wt %, preferably between 10 and 60 wt % of succinic acid. It was found advantageous that the fermentation broth comprises low amounts of salts, since this reduces the costs and process step for the recovery of such salts. As used herein, salts may be any chlorine, sulphate, phosphate, potassium, sodium, calcium, magnesium, barium, ammonium or other ions not specifically mentioned herein.

In another embodiment, the process according to the present invention further comprises removing organic components from the fermentation broth. Organic components as used herein may comprise insoluble and/or soluble organic components, such as proteins and microbial cell fragments. Removing organic components may be carried out by any method known in the art depending on the composition of the fermentation broth. Suitable methods comprise filtration, such as microfiltration, centrifugation, heat treatment, enzymatic treatment or any suitable combination of these methods. Removing organic components may comprise microfiltration, for instance microfiltration at a temperature of between 40 and 90 degrees Celsius, for instance between 50 and 80 degrees Celsius. It was found that microfiltration at this temperature range resulted in an increased yield of succinic acid in the process according to the present invention. Microfiltration may further comprise bringing a permeate from microfiltration to a temperature of between 60 and 90 degrees Celsius, for instance between 70 and 85 degrees Celsius, followed by microfiltration of the heated permeate.

In one embodiment, the process according to the present invention comprises bringing the fermentation broth to a pH of between 1 and 4, such as a pH of between 1.5 and 3.5., such as a pH of between 2 and 3. It was found that at a pH of between 1 and 4 a high yield of succinic acid crystals was achieved.

In one embodiment, said bringing the fermentation broth to a pH of between 1 and 4 comprises subjecting the fermentation broth to water-dissociation bipolar electrodialysis. Water-dissociation bipolar electrodialysis is a known technology to the skilled man in the art. Subjecting the fermentation broth to water-dissociation bipolar electrodialysis may be preceded by a step of treating the fermentation broth with a weak cation exchange resin, such as a diacetic acid or aminophosphoric chelating agent. This latter step was found advantageous to remove divalent cations, in particular Mg²⁺ and Ca²⁺ ions, which are harmful for the membranes of the bipolar electrodialyser. A step of removing organic components from the fermentation broth as outlined herein above may precede subjecting the fermentation broth to water-dissociation bipolar electrodialysis, in order to avoid pollution of the electrodialysis membrane.

Alternatively, a step of bringing the fermentation broth to a pH of between 1 and 4 comprises fermenting a fungal cell in the fermentation broth, wherein the fermenting comprises producing succinic acid. The fermenting of a fungal cell may further comprise producing other acid, for instance dicarboxylic acid, such as malic acid, fumaric acid. The production of succinic acid or other acid may continue when a desired pH value of between 1 and 4, such as between 2 and 3,5, has been reached. A neutralizing agent, such as potassium hydroxide or sodium hydroxide, may be added to the fermentation broth to maintain a desired pH value. In the event bringing the fermentation broth to a pH of between 1 and 4 comprises fermenting a fungal cell, removing of organic components from the fermentation as outlined above is preferably carried out after said bringing. A suitable fungal cell in a step of bringing a fermentation broth to a pH of between 1 and 4 may be any fungal cell as described herein below, for instance Saccharomyces cerevisiae.

A step of bringing the fermentation broth to a pH of between 1 and 4 usually further comprises a subsequent step of treating the fermentation broth with a strong acidic cation exchange resin such as a polystyrene divinylbenzene (DVB) type with sulphonic groups. Treatment of a fermentation broth with a strong cation exchange resin further increases the conversion of succinate to succinic acid with about 5 to 20%.

In one embodiment, the process for recovering of succinic acid further comprises fermenting a microbial cell in a fermentation broth to produce succinic acid. Fermenting a microbial cell usually comprises growth phase during which a microbial cell is grown to a desired cell density, and a production phase during which succinic acid is produced. The fermentation conditions during a growth phase and a (succinic) production phase may be similar or different, for instance with respect to the composition of a fermentation medium, pH or temperature.

A fermentation broth may be any suitable broth allowing growth of a microbial cell and/or production of succinic acid. The fermentation broth may comprise any suitable carbon source such as glucose, fructose, galactose, xylose, arabinose, sucrose, lactose, raffinose and glycerol. Fermenting a microbial cell may be carried out under aerobic conditions, anaerobic conditions, micro-aerophilic or oxygen limited conditions, or a combination of these fermentation conditions, for instance as disclosed in WO2009/083756. An anaerobic fermentation process is herein defined as a fermentation process run in the absence of oxygen or in which substantially no oxygen is consumed, preferably less than 5, 2.5 or 1 mmol/L/h, and wherein organic molecules serve as both electron donor and electron acceptors.

Fermenting of a microbial cell may be carried out at any suitable pH between 1 and 9, depending on the microbial cell. In the event a microbial cell is a bacterial cell, the pH in the fermentation broth preferably is between 5 and 8, preferably between 5.5 and 7.5. Usually the pH of a bacterial fermentation broth is maintained at these values by adding neutralizing agents such potassium- or sodium hydroxide, or ammonium. In the event the microbial cell is a fungal cell the pH in the fermentation broth may range between 1 and 7, preferably between 2 and 6, preferably between 2.5 and 5. The pH value during a growth phase of a fungal cell may be higher than during a (succinic acid) production phase. During fermentative production of succinic acid by a fungal cell the pH value may decrease to a pH of between 1 and 4, for instance between 2 and 3, for instance between 2.5 and 3.5. The pH during a growth phase and/or a production phase during fungal fermentation may be maintained at a desired pH value by adding a neutralizing agent.

A suitable temperature at which the fementing of a microbial cell may be carried out in the process according to the present invention may be between 5 and 60 degrees Celsius, preferably between 10 and 50 degrees Celsius, more preferably between 15 and 40 degrees Celsius, more preferably between 20° C. and 30 degrees Celsius, depending on the microbial cell. The skilled man in the art knows the optimal temperatures for fermenting a microbial cell in the process of the invention.

In one embodiment, the microbial cell is a bacterium from the genus Mannheimia, Anaerobiospirillum, Bacillus, or Escherichia, or a fungal cell from the genus Schizosaccharomyces, Saccharomyces, Aspergillus, Penicillium, Pichia, Kluyveromyces, Yarrowia, Candida, Hansenula, Humicola, Torulaspora, Trichosporon, Brettanomyces, Rhizopus, Zygosaccharomyces, Pachysolen, lssatchenkia or Yamadazyma. A bacterial cell may belong to a species Mannheimia succiniciproducens, Anaerobiospirillum succiniciproducens Bacillus amylophylus, B. ruminucola or E. coli, for instance an E. coli. A fungal cell may belong to a species Saccharomyces cervisiae, Saccharomyces uvarum, Saccharomyces bayanus, Schizosaccharomyces pombe, Aspergillus niger, Penicillium chrysogenum, P. symplissicum, Pichia stipidis, Kluyveromyces marxianus, K. lactis, K. thermotolerans, Yarrowia lipolytica, Candida sonorensis, C. glabrata, Hansenula polymorpha, Torulaspora delbrueckii, Brettanomyces bruxellensis, Rhizopus orizae, lssatchenkia orientalis or Zygosaccharomyces bailii. A fungal is for instance a yeast, for instance a Saccharomyces cerevisiae.

The microbial cell according to the present invention may be any suitable wild-type organism, or a genetically modified microorganism. Suitable genetically modified E. coli cells are disclosed in Sanchez et al., Metabolic Engineering, 7 (2005) 229-239, WO2006/031424, and U.S. Pat. No. 7,223,567. Suitable fungal cells are disclosed in WO2009/065780 and WO2009/065778.

In another preferred embodiment, a process according to the present invention may be carried out on an industrial scale. Industrial scale as used herein is defined as crystallizing succinic acid in a volume of at least 100 litres, preferably at least 1 m³, preferably at least 10 m³, more preferably at least 100, or 1000 m³, usually below 5000 m³. Succinic acid in crystal form recovered by a process according to the present invention may also be used in any suitable application, such as the production of polymers of or the production of 1,4-butanediol.

In one embodiment a process according to the invention may further comprise preparing a polyester polymer with succinic acid in crystal form. Suitable polyester polymer may be polybutylene succinate or polyester polyols. It was surprisingly found that preparing a polyester polymer with succinic acid in crystal form recovered in a process according to the present disclosure resulted in a polyester polymer such as PBS with low coloration. Low coloration is defined herein a Yellowness Index (YI) of between 0 to 100, for instance between 1 and 50, for instance between 2 and 40, or between 3 and 30.

In another aspect, the present invention relates to succinic acid in crystal form, comprising a low sugar content of between 10 to 100 ppm, preferably between 2 and 80 ppm, preferably between 4 and 60 ppm, preferably between 10 and 50 ppm, and a low nitrogen content of between 1 and 80 ppm, preferably between 2 and 60 ppm, preferably between 4 and 40 ppm. Sugar as used herein may be any suitable hexose or pentose sugar, such as glucose, galactose, xylose, arabinose, or sucrose. It was found essential that the succinic acid in crystal form comprises a low sugar content and a low nitrogen content in order to obtain succinic acid in crystal form with low coloration. Little to no coloration as used herein is defined as succinic acid crystals having an absorption at 425 nm of between 0.001 and 0.150, as measured in a solution of succinic acid in butanediol at a ratio of succinic acid in crystal form:butanediol of 1:4 which has been heated at 180 degrees Celsius for 3 hours. The succinic acid in crystal form according to the present invention is advantageously obtained by a process according to the present invention.

The succinic acid in crystal form according to the present invention surprisingly resulted in polyester polymers with low coloration.

In another aspect the present disclosure relates to a polyester polymer obtainable by a process according to the present invention. A polyester polymer may be a polybutylene succinic acid (PBS) polymer, for instance a polyester polymer having a YI of between 0 and 100, for instance between 1 and 50, for instance between 2 and 40, or a YI between 3 and 30.

### FIGURES

FIG. 1. Plasmid map of pSUC051, for overexpression of SpMAE1 and PYC2 in S. cerevisiae.

### EXAMPLES

General Materials and Methods

Color Measurement

Approximately 0.5 g succinic acid crystals were dissolved in 2 g butanediol (ratio succinic acid:butanediol of 1:4) in a capped vial and heated at 180 degrees Celsius for 3 hours. After cooling, the solution was centrifuged and the color of the supernatant was io measured at 425 nm on a spectrophotometer.

Determination Glucose

Glucose was determined by HPLC after hydrolization with HCl. The principal of the separation on a BioRad Aminex column is based on size exclusion, ion-exclusion and ion-exchange using reversed phase mechanisms. Detection took place by a differential refractive index detector.

A sample of approximately 2 g succinic acid crystals was dissolved in 2 ml distilled water and 1 ml of 2.58 mol/l HCl solution, which was incubated in a water bath at 100 degrees Celsius for 75 minutes. Subsequently, the sample was cooled to ambient temperature. The samples were analysed using the following conditions:

Conditions

| Pre-column: | Biorad Microguard Cation H+ | |
|---|---|---|
| | cartridge, 30 × 4.6 mm | |
| Column: | Biorad Aminex HPX-87H, 300 × | |
| | 7.8 mm, particle size 9 μm | |
| Column temperature: | 50° | C. |
| Flow: | 0.6 | ml/min |
| Runtime: | 12.0 | min |
| Injection volume: | 20 | μl |
| Tray temperature: | ambient | |
| RI-detector: | | |
| internal oven: | 35° | C. |

| | |
|---|---|
| time constant: | 1 |
| sensitivity: | 16 |
| scale factor: | 20 |

Determination Nitrogen

Nitrogen was determined according to Kjeldahl method.

Determination Sulphur and Cations

Sulphur and cations were determined by atomic emission spectrometry using inductively coupled plasma.

Detemination Anions

Anions such as sulfate, chloride and phosphate were separated on an anion exchange column, Dionex AS11-HC type, which was heated at 36 degrees Celsius, and detected by conductimetry. The eluent was a gradient of progressively increasing NaOH concentration. Trifluoroacetic acid was used as internal standard.

Determination Organic Acids

Organic acids were separated with ion exchange chromatography on columns (3 in series) from Biorad HPX-87H type, which were heated at 85 degrees Celsius and detected with UV at 210 nm. The elution solvent was a 5 mM sulphuric acid solution. The quantitative analysis was performed by external calibration. Pyruvic, malic, fumaric, lactic, formic and acetic acids were used as standards. The chromatographic separation was conducted in isocratic mode 60 microliters of a 3% succinic acid solution were injected.

### Example 1

### Fermentative Production of Succinic Acid in E. coli

The strain Escherichia coli SBS550MG—pHL413 (geno-type delta adhE, delta ldhA, delta icIR, delta ackpta, PYC), was prepared as disclosed in U.S. Pat. No. 7,223,567, which is herein included by reference.

1.1 Seed Culture:

Escherichia coli strain SBS550MG—pHL413 was inoculated in two 500 ml shake flasks with LB medium (Trypton 10 g/L; Yeast extract 5 g/L; NaCI 10 g/L; Ampicillin, carbenicillin, oxacillin 67 mg/L, each), which were incubated at 37° C., for 24 h. At the end of cultivation, an OD of 7 was reached

1.2. Growth Phase

Subsequently, the contents of the shake-flasks were transferred to a 1 m³ fermenter, which contained 700 L of the following medium (Table I):

TABLE I
Composition medium in fermenter

| | g/L |
|---|---|
| K₂HPO₄ | 3.6 |
| MgSO₄•7H₂O | 1.0 |
| Citric acid | 2 |
| Glucose | 5 |
| | mg/L |
| ZnSO₄•7H₂O | 30 |
| CuCl₂•2H₂O | 10 |
| MnSO₄•H₂O | 30 |
| CoCl₂•6H₂O | 1.2 |
| H₃BO₃ | 1 |
| Na₂MoO₄•2H₂O | 6 |
| Al₂(SO₄)₃•18H₂O | 7.5 |
| NiSO₄•6H₂O | 4 |
| FeSO₄•7H₂O | 90 |
| CaCl₂•2H₂O | 300 |
| Biotine | 1 |
| Thiamine | 1 |
| Ampicilline | 200 |

The pH was controlled at 6.75 by addition of 25 wt % ammonia. Temperature was controlled at 37 degrees Celsius. An airflowrate of 700 L/min was applied (1 vvm). The dissolved oxygen level (pO₂) was controlled >20% by adapting the stirrer speed. After consumption of initial 5 g/L, glucose concentration was kept limited by controlled feed of a 50 wt % solution to the fermenter. An exponential feed rate was applied. After 27 h and consumption of 25 g/L (referring to post inoculum volume) fed glucose, 10 g/L of biomass were produced (OD 30).

1.3. Transition Phase

A phase at zero pO2 (manually controlled stirring) at pH 5 by controlling with 5 N NaOH and Glucose feed at 1,6 g/L/h (refers to post inoc volume) was applied during 6.5 h.

1.4. Production Phase

This phase was started by switching to 0.1 vvm CO₂ supply at 0.1 vvm and adding 75 g/L of glucose (ref post inoc volume). pH and temperature were controlled at 6.75 and 37 degrees Celsius respectively. After 93h of fermentation, 45 g/L of succinic acid was obtained. The composition of the fermentation broth at the end of fermentation (besides biomass) is shown in Table II.

TABLE II
Composition of the E. coli fermentation broth

| | Impurities relative to succinic acid |
|---|---|
| Compounds | (% weight/weight) |
| Succinic acid | 100 |
| Fumaric acid | 0.08 |
| Citric acid | 10.38 |
| Lactic acid | 1.08 |
| Pyruvic acid | 0.12 |
| Formic acid | 3.65 |
| Acetic acid | 1.68 |
| Orotic acid | 0.62 |
| Malic acid | 0.50 |
| Organic nitrogen | 2.47 |
| NH₄ | 1.18 |
| Na | 45.91 |
| K | 2.40 |
| Mg | 0.14 |
| Ca | 0.10 |
| Fe | 0.02 |
| Sulphates | 0.26 |
| Phosphates | 2.59 |
| Bicarbonates | 0.04 |
| Chlorides | 0.23 |

### Example 2

### Recovery of Succinic Acid from E. coli Fermentation Broth

2.1. Removal of Biomass

The fermentation broth obtained in Example 1 was filtered by tangential filtration with tangential flow on a ceramic membrane with a channel diameter of 3.5 mm, having a pore diameter of 100 nm, with a trans-membrane pressure of 1 bar resulting in a mean flow of 90 l/h/m². The temperature was maintained at 60° C.

2.2. Removal of Soluble Organic Impurities: The Residual Soluble Proteins

The fermentation broth obtained from step 2.1 described above was heated to 80° C. for 10-15 min, which allowed flocculation of proteins. The solution was subsequently filtered through a filter with a pore diameter of 0.22 μm.

2.3. Cation Exchange and Water-Dissociation Bipolar Electrodialysis

The solution from step 2.2. was treated with a weak cation exchange resin,

Amberlite IRC747. The solution from step 2.2 was supplied at a flow rate of 2 Bed Volumes (BV)/h at 60° C. The cation exchange treatment reduced the amount of divalent ions (Ca²⁺ and Mg²⁺) to below 5 ppm.

The solution treated with cation exchange resin was treated with water-dissociation bipolar electrodialysis, EUR6 from Eurodia. The flow of cations was approximately 22 eq/h/m², converting 90% of succinate to succinic acid. The pH at the end of the electrodialysis was about 3.5. For reasons of energy saving, the degree of conversion was fixed at 90%.

The solution treated by water-dissocation bipolar electrodialysis was treated with a strong cationic resin (Purolite C150). The cationic resin treatment was done at 40° C. and at 2 BV/h. The volume of the solution treated was approximately 10-15 bed volumes. The pH of the solution after treatment on cationic resin was 2.

2.4. Crystallization

The acidified solution obtained in step 2.3. was concentrated by evaporation of water on a Wiegand® falling-film evaporator at 80 degrees Celsius, until supersaturation was reached (approximately 420 g/l). Subsequently, the solution was cooled at a rate of 5 degrees Celsius/h from 80 to 20 degrees Celsius and succinic acid was crystallized.

After separation on a ROUSSELET® centrifuge and after washing with one volume of demineralized water per cake volume, the succinic acid crystals were dried.

At this step, the yield of succinic acid was >85% with a purity of 99.7%.

The composition of these succinic acid crystals is presented in Table III below.

TABLE III
Composition of succinic acid crystals

| | Impurities relative to succinic acid |
|---|---|
| Compounds | (% weight/weight) |
| Succinic acid | 100 |
| Fumaric acid | 0.17 |
| Citric acid | 0.03 |
| Lactic acid | <0.10 |
| Pyruvic acid | <0.02 |
| Formic acid | <0.10 |
| Acetic acid | <0.10 |
| Orotic acid | 0.02 |
| Malic acid | <0.10 |
| Organic nitrogen | 0.02 |
| NH₄ | <0.01 |
| Na | <0.01 |
| K | <0.01 |
| Mg | <0.01 |
| Ca | <0.01 |
| Fe | <0.01 |
| Sulphates | <0.01 |
| Phosphates | 0.01 |
| Bicarbonates | 0 |
| Chlorides | <0.01 |

2.5. Dissolution

The crystals obtained in example 2.4 were dissolved in demineralized water at 60° C.

2.6. Decolouring

An amount of Norit SX⁺ activated carbon of 1 wt % of succinic acid was added to the dissolved succinic acid solution obtained in step 2.5. After 1 h at 60° C., the solution was filtered on a 3 μm candle filter.

Subsequently, the solution was demineralized at 2 BV/h at 60° C. on a strong cationic resin Purolite® C150 and weak anionic resin Amberlite FPA55. The treatment allowed removal of the traces of mineral cations and anions and of fumaric acid and traces of orotic acid (when present). Moreover, the weak anionic resin allowed additional decolourizing.

2.7. Crystallization

The solution purified under step 2.7., was crystallized under the same conditions as described in example 2.4.

The composition of the high-purity crystals is given in Table IV below:

TABLE IV
Composition of the high-purity crystals from
E. coli succinic acid fermentation process

| | Impurities relative to succinic acid |
|---|---|
| Compounds | (% weight/weight) |
| Succinic acid | 100 |
| Fumaric acid | <0.01 |
| Citric acid | <0.01 |
| Lactic acid | <0.01 |
| Pyruvic acid | <0.01 |
| Formic acid | <0.01 |
| Acetic acid | <0.01 |
| Orotic acid | <0.005 |
| Malic acid | <0.1 |
| Organic nitrogen | <0.01 |
| NH₄ | <0.01 |
| Na | <0.01 |
| K | <0.01 |
| Mg | <0.01 |
| Ca | <0.01 |
| Fe | <0.01 |
| Sulphates | <0.01 |
| Phosphates | <0.01 |
| Bicarbonates | 0 |
| Chlorides | <0.01 |

The yield of this crystallization step was likewise about 85%.

The overall yield of succinic acid after this final crystallization without recycle was about 65 wt % of the succinic acid originally present in the fermentation broth.

### Example 3

### Succinic Acid Fermentation Process of Saccharomyces cerevisiae at pH 5

Yeast strain S. cerevisiae SUC-200, RWB064 (CEN.PK113-6B, MATA ura3-52 leu2-112 trp1-289 adh1::lox adh2::lox gpd1::Kanlox), overexpressing PCKa, MDH3, FUMR, FRDg and SpMAE1 was prepared as disclosed in WO2009/065778 p. 23-34 including the corresponding sequence listings, which is herein incorporated by reference. Strain SUC-200 was cultivated in shake-flasks (2×300 ml) at 130 rpm, at 30 degrees Celsius 3 days, in a medium shown in Table V. The final OD at 620 nm was 26.

Subsequently, the contents of the shake-flasks were transferred to a 10 L fermenter (Startweight 6 kg), which contained a medium shown in Table VIII.

The pH was controlled at 5.0 by addition of 6N KOH . The temperature was controlled at 30° C. An airflowrate of 2 L/min was applied (0.3 vvm). The dissolved oxygen level (pO₂) was controlled at about 20% by adapting the stirrer speed.

After 16.5 h of growth, an OD at 620 nm of 35 was reached and the culture was transferred to a 1 m³ fermenter (startweight 700 kg), which contained the medium shown in Table IX.

TABLE V
Preculture shake flask medium composition

| Raw material | Concentration (g/l) |
|---|---|
| Galactose (C₆H₁₂O₆•H₂O) | 20.0 |
| Urea ((NH₂)₂CO) | 2.3 |
| KH₂PO₄ | 3.0 |
| MgSO₄•7H₂O | 0.5 |
| Trace element solution^{b} | 1 |
| Vitamin solution^{a} | 1 |

TABLE VI
^{a}Vitamin solution

| | | Concen- |
|---|---|---|
| Component | Formula | tration (g/kg) |
| Biotin (D−) | C₁₀H₁₆N₂O₃S | 0.05 |
| Ca D(+) panthothenate | C₁₈H₃₂CaN₂O₁₀ | 1.00 |
| Nicotinic acid | C₆H₅NO₂ | 1.00 |
| Myo-inositol | C₆H₁₂O₆ | 25.00 |
| Thiamine chloride hydrochloride | C₁₂H₁₈Cl₂N₄OS × H₂O | 1.00 |
| Pyridoxol hydrochloride | C₈H₁₂ClNO₃ | 1.00 |
| p-aminobenzoic acid | C₇H₇NO₂ | 0.20 |

TABLE VII
^{b}Trace elements solution

| Component | Concentration (g/kg) |
|---|---|
| EDTA | 15.00 |
| ZnSO₄•7H₂O | 4.50 |
| MnCl₂•2H₂O | 0.84 |
| CoCl₂•6H₂O | 0.30 |
| CuSO₄•5H₂O | 0.30 |
| Na₂MoO₄•2H₂O | 0.40 |
| CaCl₂•2H₂O | 4.50 |
| FeSO₄•7H₂O | 3.00 |
| H₃BO₃ | 1.00 |
| KI | 0.10 |

TABLE VIII
Medium composition growth fermenter

| | | Concentration |
|---|---|---|
| Raw material | | (g/l) |
| Galactose | | 20 |
| Ammonium sulphate | (NH₄)₂SO₄ | 1 |
| Potassium dihydrogen phosphate | KH₂PO₄ | 10 |
| Magnesium sulphate | MgSO₄•7H₂O | 5 |
| Trace element solution | | 8 |
| Vitamin solution | | 8 |

TABLE IX
Medium composition production fermenter

| | | Concentration |
|---|---|---|
| Raw material | | (g/l) |
| Ammonium sulphate | (NH₄)₂SO₄ | 2.5 |
| Potassium dihydrogen phosphate | KH₂PO₄ | 5 |
| Magnesium sulphate | MgSO₄•7H₂O | 0.5 |
| Trace element solution | | 1 |
| Vitamin solution | | 1 |

The pH was controlled at 5.0 by addition of 6 N KOH. The temperature was controlled at 30° C. The glucose concentration was kept limited by controlled feed of a 50 wt % solution to the fermenter. When glucose accumulated, the feedrate was decreased stepwise as shown below.

Actual glucose feed profile during the fermentation

| Time (h) | Setpoint (g/h) |
|---|---|
| 0 | 350 |
| 20 | 355 |
| 50 | 1050 |
| 90 | 1050 |

Feed increased linearly between points

During the first 20 h, air was supplied at 0.3 vvm and pO₂ was controlled at about 20% by adapting the stirrer speed.

After 20 h, an OD at 620 nm of 8 was reached and gaz inlet was changed to a mix including 50% CO₂ to supply enough CO₂ for efficient succinic acid production. Total gazflow was maintained at 0.33 vvm and the oxygen uptake rates (OUR) were controlled at 5-8 mmol/l/h by adapting the stirrer speed.

After 145 h of fermentation, 34.5 g/L of succinic acid was obtained. By-product included 4.5 g/L ethanol, 7.7 g/L glycerol and 7.8 g/L malic acid.

### Example 4

### Recovery of Succinic Acid from S. cerevisiae Fermentation Broth at pH 5

After fermentation of S. cerevisiae SUC-200 strain as described above, the biomass was removed by filtration as described in Example 2.1. The filtered broth had a composition as described in Table X.

TABLE X
Composition of the S. cerevisiae fermentation broth

| | Impurities relative to succinic acid |
|---|---|
| Compounds | (% weight/weight) |
| Succinic acid | 100 |
| Fumaric acid | 1.14 |
| Citric acid | <0.15 |
| Lactic acid | 0.9 |
| Pyruvic acid | 0.51 |
| Formic acid | <1.0 |
| Acetic acid | 4.49 |
| Orotic acid | 0.14 |
| Malic acid | 22.26 |
| Ethanol | 12.9 |
| Glycerol | 22.0 |
| Organic nitrogen | 2.28 |
| NH₄ | 0.046 |
| Na | 0.25 |
| K | 61.4 |
| Mg | 0.09 |
| Ca | <0.03 |
| Sulphates | 4.14 |
| Phosphates | 2.83 |
| Bicarbonates | <0.06 |
| Chlorides | <0.015 |

4.1. Removal of Organic Impurities and Acidification

Organic impurities were removed from the filtered broth as described in Example 2.2.

Subsequently, the filtered solution was acidified by cation exchange resin and water-dissociation bipolar electrodialysis as described in Example 2.3.

4.2. Crystallization of Succinic Acid

The solution having a pH 2, as obtained in Example 4.1 was concentrated and crystallized in an adiabatic crystallizer, as described in example 2.4. The broth was cooled from 80 degrees Celsius to 25 degrees Celsius, by applying a vacuum control slope (from 500mb to 35 mb).

Subsequently, crystals were removed and washed as described in example 2.4. The yield of succinic acid crystals of this crystallization step was about 85% with a purity of about 98.5%.

4.3. Purification of Succinic Acid Crystals:

The succinic acid crystals obtained in example 4.2. were dissolved, treated with activated carbon and crystallized as described in Example 2.5, 2.6 and 2.7.

Separation and washing was performed on the same equipment but with 0.5 kg of water/kg of crystals.

After drying at 60° C., during 10 minutes on a fluidized bed, high-purity succinic acid crystals were obtained (see Table XI below).

TABLE XI
Composition of the succinic acid crystals obtained
after pH 5 S. cerevisiae fermentation process

| Compounds | Amount (%/solids) |
|---|---|
| Succinic acid | 100 |
| Fumaric acid | <0.01 |
| Citric acid | <0.05 |
| Lactic acid | <0.1 |
| Pyruvic acid | <0.01 |
| Formic acid | <0.1 |
| Acetic acid | <0.1 |
| Orotic acid | <0.005 |
| Malic acid | <0.1 |
| Ethanol | <1.5 |
| Glycerol | <0.2 |
| Organic nitrogen | <0.006 |
| NH₄ | <0.01 |
| Na | <0.01 |
| K | <0.01 |
| Mg | <0.01 |
| Ca | <0.01 |
| Fe | <0.01 |
| Sulphates | <0.01 |
| Phosphates | <0.01 |
| Chlorides | <0.01 |

### Example 5

### Succinic Acid Fermentation Process of Saccharomyces cerevisiae at pH 3

For the fermentation process at pH 3, S. cerevisiae strain SUC-297 was used which was constructed as described herein below.

5.1. Gene Sequence

Cytoplasmic pyruvate carboxylase from Saccharomyces cerevisiae (Pyc2p) [E.C. 6.4.1.1.], GenBank accession number 1041734, was codon-pair optimized for io expression in S. cerevisiae as disclosed in WO2008/000632. In the synthetic gene sequence, the stop codon TAA was modified to TAAG. The synthetic PYC2 gene is under control of (or operable linked to) a strong promoter from S. cerevisiae, i.e. the PGK1-promoter (600 by upstream of the start codon of the PGK1 gene). Proper termination is controlled by a terminator sequence from S. cerevisiae, i.e. the PGK1-terminator (300 by downstream of the stop codon of the PGK1 gene). The PGK1-promoter;PYC2-gene;PGK1-terminator sequence was amongst others surrounded by the unique restriction enzymes sites HindIII at the 5′ end and BamHl at the 3′ end of the synthetic gene sequence. The resulting sequence SEQ ID NO: 1 was synthesised by Geneart (Regensburg, Germany).

5.2. Construction of Expression Plasmids

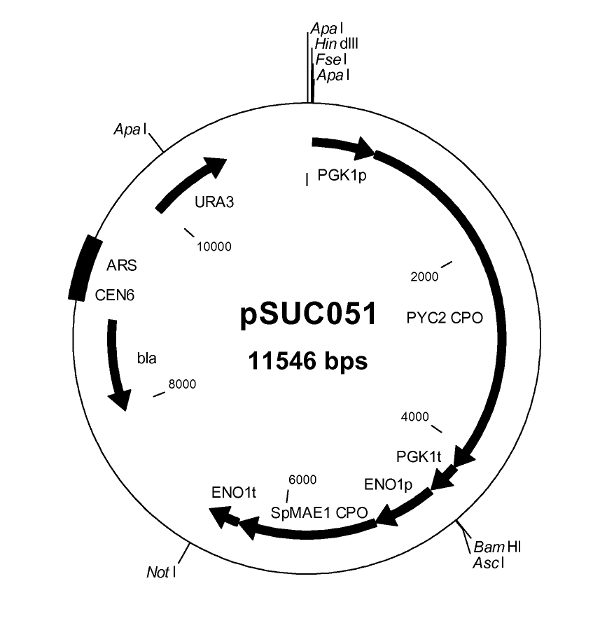

Plasmids pGBS414PPK-3 and pGBS415FUM-3 were prepared as disclosed in WO2009/065778. Plasmid pSUC051, as set out in FIG. 1, was constructed as follows: Plasmid pGBS416MAE-1, prepared as disclosed in WO2009/065778, Example 2D, FIG. 18, was restricted with the restriction enzymes HindIII and BamHI. Subsequently, a HindIII/BamHI restriction fragment consisting of the PGK1p-PYC2-PGK1t synthetic gene construct (SEQ ID NO: 1) was ligated in the restricted plasmid. The ligation mix was used for transformation of E. coli TOP10 (Invitrogen) resulting in the yeast expression construct pSUC051 (FIG. 1).

5.3. Construction of Yeast Strain SUC-297

Yeast strain S. cerevisiae SUC-297, RWB064 (CEN.PK113-6B, MATA ura3-52 leu2-112 trp1-289 adh1::lox adh2::lox gpd1::Kanklox), overexpressing PCKa, MDH3, FUMR, FRDg, SpMAE1 and PYC2 from episomal plasmids was prepared as disclosed in WO2009/065778 p. 23-34 including the corresponding sequence listings, which is herein incorporated by reference, with the following exception: Instead of transforming RWB064 with pRS416MAE-1, (in addition to pGBS414PKK-3 and pGBS415FUM-3 for the construction of SUC-200), RWB064 was transformed with pSUCO51 (for overexpression of SpMAE1 and PYC2), pGBS414PPK-3 (for overexpression of PCKa and FRDg, FIG. 11 in WO2009/065778), and pGBS415FUM-3 (for overexpression of MDH3 and FUMR, FIG. 16 in W02009/065778) resulting in strain SUC-297.

5.4. S. cerevisiae Succinic Acid Fermentation Process at pH 3

Yeast strain S. cerevisiae SUC-297 as described above in example 5.1-5.3 was cultivated in shake-flasks (3×2 liter) at 140 rpm, at 30 degrees Celsius during 3 days, in a similar medium shown in Table V, with the exception that the concentration of galactose was 21 g/l, urea: 2.415 g/l, KH₂PO₄:3.15 g/l, and MgSO₄.7H₂: 0.525 g/l. The final OD at 600 nm was between 17 and 20.

Subsequently, the contents of the shake-flasks were transferred to a 70 L fermenter (Startweight 67 kg), which contained a similar medium as shown in Table VIII, with the exception that the medium did not contain galactose. Instead, glucose (sterilized glucose 50 w/v % in water) was fed during the growth fermentation in an exponential feed (0.2*exponent (0.1*time)) with a limit of 10 g/L/hour.

The pH during growth fermentation was controlled at 5.0 by addition of 28% NH₃. The temperature was controlled at 30° C. An airflowrate of 70 L/min was applied (1 vvm). The dissolved oxygen level (p0₂) was controlled at about >20% by adapting the stirrer speed.

Growth was continued until an OD at 600 nm of 300 (after several days of cultivation) the culture was transferred to a 1 m³ fermenter (startweight 629 kg), which contained a similar medium as shown in Table IX, with the exception that 3 g/l KH₂PO₄ was used instead of 5 g/l.

The pH was controlled at 5.0 by addition of 50% KOH in water. After 4 hours the pH control was changed to a set point of 3.0, resulting in a reduction in pH in the fermentation broth to 3.0. The temperature was controlled at 30° C. The glucose concentration was kept limited by controlled feed of a 50 wt % solution to the fermenter. When glucose accumulated, the feedrate was decreased stepwise.

The fermenter was aerated with a gas mixture of air and 50% CO₂ to supply enough CO₂ for efficient succinic acid production. Total gasflow was maintained at 0.33 vvm and the oxygen uptake rates (OUR) were controlled at 5-8 mmol/l/h by adapting the stirrer speed.

After 95 h of fermentation, 43 g/L of succinic acid was produced. By-products included 16.4 g/L ethanol and 14.9 g/L glycerol.

5.5. Recovery of Succinic Acid from pH 3 Fermentation Broth.

The fermentation broth obtained in Example 5.4 was collected and yeast cells were removed by microfiltration (0.1 micron KERASEP membrane), yielding a clarified filtrate. The clear filtrate was subsequently contacted with a strong acidic cation exchange resin (Purolite C150) to remove the cationic minerals and reduce the pH. After removal of the ion exchange resin (by filtration) the pH of the filtrate was <2. The acidified broth filtrate was subsequently concentrated by evaporation, crystallized and re-crystallized according to the method described in example 2.4 to 2.7. Highly purified succinic acid crystals were obtained as shown in table XII.

TABLE XII
Composition high purity crystals obtained after
pH 3 S. cerevisiae fermentation process

| | Unit (on dry | Highly purified succinic |
|---|---|---|
| Compounds | weight basis) | acid crystals |
| Succinic acid | % wt | 100 |
| Fumaric acid | % wt | 0.003 |
| Citric acid | % wt | N.D. |
| Lactic acid | % wt | <0.1 |
| Pyruvic acid | % wt | <0.01 |
| Formic acid | % wt | <0.01 |
| Acetic acid | % wt | <0.01 |
| Orotic acid | ppm | <50 |
| Malic acid | % wt | <0.01 |
| Total nitrogen | ppm | 2.7 |
| NH₄ | ppm | <50 |
| Sulphates | ppm | <50 |
| Phosphates | ppm | <50 |
| Bicarbonates | ppm | <50 |
| Chlorides | ppm | <50 |

### Example 6

### Color, Glucose and Nitrogen Content

The color, glucose and nitrogen content of succinic acid crystals obtained from E. coli fermentation (Example 1 and 2), and from S. cerevisiae fermentation (Example 3 and 4), were determined as described above. The results are shown in Table XIII.

TABLE XIII
Color, sugar and nitrogen content in samples of succinic acid crystals
obtained from E. coli (BE) and S. cerevisiae (BY) fermentations.

| | | Color | Glucose | Nitrogen |
|---|---|---|---|---|
| No. | | (425 nm) | ppm (mg/kg) | ppm (mg/kg) |
| | E. coli | | | |
| | Succinic acid crystals | | | |
| 1 | BE20 | 0.031 | 1-10 | 1 |
| 2 | BE24 | 0.067 | 1-10 | 1 |
| 3 | BE25 | 0.060 | 1-10 | 1 |
| 4 | BE30 | 0.015 | 1-10 | 1 |
| | S. cerevisiae | | | |
| | Succinic acid crystals | | | |
| 5 | BY2 | 0.118 | 37 | 1 |
| 6 | BY4 | 0.014 | 1-10 | 5 |
| 7 | BY6 | 0.002 | 1-10 | 7 |

Conclusion

The results show that a process for recovering succinic acid in crystal form according to the present invention resulted succinic acid in crystal form of high purity both from an E. coli and S. cerevisiae fermentation broth. Acidifying a fermentation medium with EBDM or fermenting S. cerevisiae resulted in a similar quality of succinic acid crystals

### Example 7

### Preparation of Polybutylene Succinate (PBS) and Color Measurements

Succinic acid samples from S. cerevisiae were used in the preparation of PBS.

7.1. Polycondensation

PBS was prepared by polycondensation in the following way:

1 equivalent succinic acid and 1.2 equivalents 1,4-butanediol (99.9%) were esterified at 220 degrees Celsius with Ti-butoxide as catalysator (60 ppm Ti/kg polyester). The first condensation step is conducted under nitrogen at ambient pressure until more than 90% of the theoretical amount of water is distilled off. The second step (transesterification) is performed in vacuo (0.2-0.4 mbar) to distill off the excess of 1,4-butanediol and to build up molecular weight. The polycondensations ran for 4-5 hours. Molecular weights of the PBS samples were in the range of Mn=12 000 g/mol (by GPC).

7.2. Color Measurement

Polybutylenesuccinic acid (PBS) obtained as described above was pressed into plates at 140° C./10 min in a melt. press. The color of samples from S. cerevisiae crystals obtained as described in examples 3 and 4 and the PBS plates were measured with a Dr. Lange Micro Color II instrument. Succinic acid was ground and measured as fine powder. Plates were measured with the white standard plate as background. All measurements io were done in triplicate on 3 different spots and averaged. YI (Yellowness Index) and WI (Whiteness Index) were calculated and given according to ASTM 313. The results are shown in Table XIV.

TABLE XIV
YI and WI values of succinic acid (SA) powder recovered
from S. cerevisiae and PBS made therefrom

| | |
|---|---|
| SA powder | PBS |

| Sample | YI | WI | YI | WI |
|---|---|---|---|---|
| BYxx | 0.0 | 87.5 | 9.2 | 53.6 |
| BY6 | 1.8 | 80.7 | 18.0 | 30.4 |

The results in Table XIV show that succinic acid produced with S. cerevisiae showed little coloration and high quality PBS was produced with these succinic acid crystals

1. A process for recovering succinic acid in crystal form from a fermentation broth comprising succinic acid, comprising:
a) bringing said fermentation broth to a pH of from 1 to 4,
b) crystallizing said succinic acid from said fermentation broth to form succinic acid crystals,
c) dissolving said succinic acid crystals at a temperature of from 30 to 100 degrees Celsius to form an aqueous solution comprising dissolved succinic acid,
d) crystallizing said succinic acid from the solution to recover said succinic acid in crystal form.

2. The process according to claim 1, further comprising decoloring said solution comprising dissolved succinic acid crystals.

3. The process according to claim 2 wherein the decoloring comprises treating said solution comprising said dissolved succinic acid crystals with activated carbon.

4. The process according to claim 1, wherein said fermentation broth comprises at least one salt component at a concentration of 1 to 80 wt % of said succinic acid.

5. The process according to claim 1, further comprising removing at least one organic component from said fermentation broth.

6. The process according to claim 1, wherein said bringing comprises subjecting said fermentation broth to water-dissociation bipolar electrodialysis.

7. The process according to claim 1, wherein said bringing comprises fermenting a fungal cell in said fermentation broth, wherein said fermenting comprises producing said succinic acid.

8. The process according to claim 7, wherein said fungal cell is Saccharomyces cerevisiae.

9. The process according to claim 1, wherein said process comprises fermenting a microbial cell in said fermentation broth to produce said succinic acid.

10. The process according to claim 9, wherein said microbial cell is an Escherichia coli.

11. The process according to claim 9, wherein said microbial cell is a Saccharomyces cerevisiae.

12. The process according to claim 1, further comprising preparing a polyester polymer with said succinic acid in crystal form.

13. The process according to claim 1, wherein said process is carried out on an industrial scale.

14. Succinic acid in crystal form, comprising a sugar content of 1 to 100 ppm and a nitrogen content of 1 to 80 ppm.

15. Succinic acid in crystal form according to claim 13, comprising an absorption at 425 nm of from 0.001 to 0.150, as measured in a solution of succinic acid in butanediol at a ratio of succinic acid:butanediol of 1:4 which has been heated at 180 degrees Celsius for 3 hours.

16. Succinic acid in crystal form comprising a sugar content of 1 to 100 ppm and a nitrogen content of 1 to 80 ppm, obtainable by said process according to claim 1.

17. A polyester polymer obtainable by said process according to claim 12.

18. A polyester polymer according to claim 17, comprising a YI of from 1 to 100.
